(19) United States
(12) Patent Application Publication
(10) Pub. No.: US 2011/0040360 A1
(43) Pub. Date: Feb. 17, 2011

(54) PAIN-RELIEF WRAP

(76) Inventor: Norlita Subia, Federal Way, WA (US)

Correspondence Address: LAW OFFICE OF JERRY D. HAYNES , P.A, P.O. Box 350392, Fort Lauderdale, FL, 33335, US

(21) Appl. No.: 12/541,286

(22) Filed: Aug. 14, 2009

(51) Int. Cl. A61F 7/02

(52) U.S. Cl. 607/112; 607/114

(57) ABSTRACT

A pain-relief wrap comprising a plurality of pouch members and a pair of elastic members. Each pouch member comprises a first end portion, a second end portion, a pouch section enclosing at least one pain relieving medium, a first loop, and a second loop. Each of the pair of elastic members comprises a proximal end portion and a distal end portion. The proximal end portion and the distal end portion of a first elastic member of the pair of elastic members are coupled to each other on reception of the first elastic member through the first loop of each of the plurality of pouch members, and, the proximal end portion and the distal end portion of a second elastic member of the pair of elastic members are coupled to each other on reception of the second elastic member through the second loop of each of the plurality of pouch members.

### FIELD OF THE DISCLOSURE

The present disclosure relates generally to pain relief apparatuses, and more particularly, to a pain-relief wrap for application to an affected part of a person.

### BACKGROUND OF THE DISCLOSURE

Ice packs and heat packs are usually employed for relieving pain in affected parts of a person. The ice packs may be applied to an affected part, such as a bruised part of the person. The application of the ice packs to the bruised part absorbs heat from the bruised part. Such absorption of heat limits the amount of swelling in the bruised part, and alleviates the discomfort of the person.

Further, the heat packs may be applied to an affected part, such as a strained part of the person. The application of the heat packs transfers heat to the sprained part. Such transfer of heat to the strained part limits the amount of swelling in the sprained part, and alleviates the discomfort of the person.

However, conventional ice packs and heat packs (hereinafter referred to as pain-relief packs) are inconvenient to use and suffer from limitations. Firstly, conventional pain-relief packs include complex attachments. Secondly, the conventional pain-relief packs are required to be assembled before use. Thirdly, the conventional pain-relief packs require the person to perform the cumbersome activity of holding the conventional pain-relief packs in contact with the affected parts for long periods of time.

Further, the conventional pain-relief packs are bulky. As a result, the application of the conventional pain-relief packs to the affected parts of the person restricts the movements of those parts. Such restriction in the movement of the affected parts hampers daily activities of the person, especially when the affected parts are arms and legs of the person.

Therefore, there exists a need for a pain-relief pack, which is simple in construction. Further, the pain-relief pack should be easy to assemble, thereby making it convenient to use. Moreover, the pain-relief pack should be capable of removably securing against an affected part of the person. In addition to the above, the pain-relief pack should be lightweight and compact so that it does not restrict the movement of the affected part of the person.

### SUMMARY OF THE DISCLOSURE

In view of the foregoing disadvantages inherent in the prior art, the general purpose of the present disclosure is to provide a pain-relief pack in form of a pain-relief wrap. The pain-relief wrap includes all the advantages of the prior art, and overcomes the drawbacks inherent therein.

Accordingly, an object of the present disclosure is to provide a pain-relief wrap, which is capable of removably securing itself against the affected part of the person.

Another object of the present disclosure is to provide a lightweight and compact pain-relief wrap such that it does not restrict the movement of the affected part of the person.

Yet another object of the present disclosure is to provide a pain-relief wrap, which is simple in construction and easy to use.

In light of the above objects, disclosed is a pain-relief wrap for application to an affected part of a person. The pain-relief wrap comprises a plurality of pouch members and a pair of elastic members. Each pouch member of the plurality of pouch members comprises a first end portion, a second end portion opposite to the first end portion, a pouch section configured between the first end portion and the second end portion and capable of enclosing at least one pain relieving medium therein, a first loop configured at the first end portion, and a second loop configured at the second end portion. Each elastic member of the pair of elastic members comprises a proximal end portion and a distal end portion. A first elastic member of the pair of elastic members is received through the first loop of each pouch member of the plurality of pouch members, and, the proximal end portion and the distal end portion of the first elastic member are coupled to each other on receiving the first elastic member through the first loop of the each pouch member. A second elastic member of the pair of elastic members is received through the second loop of the each pouch member, and, the proximal end portion and the distal end portion of the second elastic member are coupled to each other on receiving the second elastic member through the second loop of each of the plurality of pouch members. The pair of elastic members is capable of removably securing the pain-relief wrap to the affected part of the person on application of the pain-relief wrap to the affected part of the person.

The pain-relief wrap configured in such a manner has bracelet-like design and may be slidably received over an arm portion or a leg portion of the person for application to the affected part of the person. Moreover, the pair of elastic members is capable of removably securing the pain-relief wrap to the affected part of the person, thereby precluding the cumbersome activity of holding the at least one pain relieving medium in contact with the affected part. Furthermore, the pain-relief wrap is configured to be lightweight and compact such that it does not restrict the movement of the affected part of the person. Further, as described above, the pain-relief wrap is simple in construction and easy to use.

This together with other embodiments of the present disclosure, along with the various features of novelty that characterize the present disclosure, are pointed out with particularity in the claims annexed hereto and form a part of this disclosure. For a better understanding of the present disclosure, its operating advantages, and the specific objects attained by its uses, reference should be made to the accompanying drawings and the descriptive matter in which there are illustrated exemplary embodiments of the present disclosure.

### BRIEF DESCRIPTION OF THE DRAWINGS

The advantages and features of the present disclosure will become better understood with reference to the following detailed description and claims taken in conjunction with the accompanying drawings, in which:

FIG. 1 depicts a perspective view of a pain-relief wrap for application to an affected part of a person, in accordance with an embodiment of the present disclosure;

FIG. 2 depicts a perspective view of a pouch member of the plurality of pouch members of the pain-relief wrap of FIG. 1, in accordance with an embodiment of the present disclosure;

FIG. 3A depicts a perspective view of the pain-relief wrap removably secured to an arm portion of a person, in accordance with an exemplary embodiment of the present disclosure; and

FIG. 3B depicts a perspective view of the pain-relief wrap removably secured to a leg portion of a person, in accordance with an exemplary embodiment of the present disclosure.

Like reference numerals refer to like parts throughout the description of the drawings.

### DETAILED DESCRIPTION OF THE DISCLOSURE

The exemplary embodiments described herein detail for illustrative purposes are subject to many variations in structure and design. It should be emphasized, however, that the present disclosure is not limited to a particular pain-relief wrap, as shown and described. It is understood that various omissions and substitutions of equivalents are contemplated as circumstances may suggest or render expedient, but these are intended to cover the application or implementation without departing from the spirit or scope of the claims of the present disclosure. Also, it is to be understood that the phraseology and terminology used herein is for the purpose of description and should not be regarded as limiting. The terms “first,” “second,” and the like, herein do not denote any order, quantity, or importance, but rather are used to distinguish one element from another, and the terms “a” and “an” herein do not denote a limitation of quantity, but rather denote the presence of at least one of the referenced item.

The use of “including,” “comprising,” or “having” and variations thereof herein is meant to encompass the items listed thereafter and equivalents thereof as well as additional items. Unless limited otherwise, the terms “configured,” “disposed,” and variations thereof herein are used broadly and encompass direct and indirect attachments, couplings, and engagements. In addition, the terms “attached” and “coupled” and variations thereof are not restricted to physical or mechanical attachments or couplings.

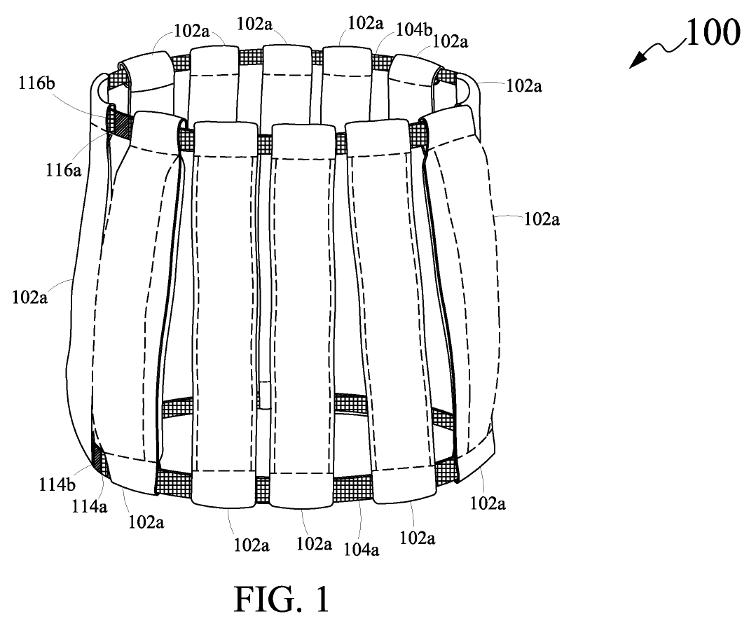

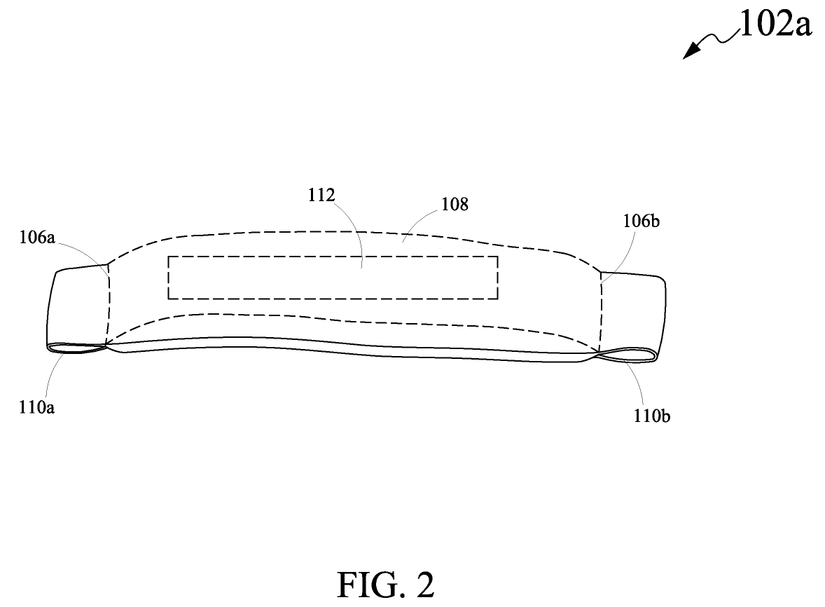

The present disclosure relates to a pain-relief wrap. The disclosed pain-relief wrap is useful for application to an affected part of a person. It should be understood that the term ‘affected part’ as mentioned herein refers to a bruised part, a sprained part, or any other injured part of the person that requires ice therapy or heat therapy. Further, it should be understood that such part may be a portion of an arm, a wrist portion, a leg portion, ankles, knees or thighs of the person. The pain-relief wrap will now be explained in detail in conjunction with FIG. 1 and FIG. 2.

FIG. 1 depicts a perspective view of a pain-relief wrap 100 for application to an affected part (not shown) of a person (not shown), in accordance with an embodiment of the present disclosure. The pain-relief wrap 100 includes a plurality of pouch members, such as a pouch member 102a, and a pair of elastic members. The pair of elastic members includes a first elastic member 104a and a second elastic member 104b. Pouch members, such as the pouch member 102a, will hereinafter be collectively referred to as plurality of pouch members. The pair of elastic members, i.e. the first elastic member 104a and the second elastic member 104b, will hereinafter be collectively referred to as pair of elastic members. As shown in FIG. 1, the pain-relief wrap 100, configured using the plurality of pouch members and the pair of elastic members, has a bracelet-like design and may be slidably received over an arm portion or a leg portion of the person for application to the affected part of the person. Further, each pouch member of the plurality of pouch members includes at least one pain relieving medium, capable of providing relief from pain in the affected part of the person. A pouch member, such as the pouch member 102a, is explained in conjunction with FIG. 2.

FIG. 2 depicts a perspective view of the pouch member 102a of the plurality of pouch members of the pain-relief wrap 100, in accordance with an embodiment of the present disclosure. It will be evident to those skilled in the art that the pouch member 102a is depicted for exemplary purposes and that each pouch member of the plurality of pouch members of the pain-relief wrap 100 is similar to the pouch member 102a. Further, the description for the each pouch member is not included herein for the sake of brevity of the description of the pain-relief wrap 100.

The pouch member 102a includes a first end portion 106a, a second end portion 106b opposite to the first end portion 106a, a pouch section 108 configured between the first end portion 106a and the second end portion 106b, a first loop 110a configured at the first end portion 106a and a second loop 110b configured at the second end portion 106b. The pouch section 108 is capable of enclosing at least one pain relieving medium, such as a pain relieving medium 112, therein.

The pain relieving medium 112 is capable of relieving pain in the affected part of the person. The term ‘pain-relieving medium’ as mentioned herein may refer to any icing material that may be employed for cooling a surface. A suitable example of such icing material may include an ice gel, or any other synthetic coolant capable of cooling a surface. In an exemplary embodiment of the present disclosure, the pain relieving medium 112 is ice gel in form of a ‘Rubbermaid Blue Ice®’ gel pack. Moreover, it should be understood that the pouch member 102a may include different units of ‘Rubbermaid Blue Ice®’ gel packs. For example, the pouch member 102a may include one unit of ‘Rubbermaid Blue Ice®’ gel pack for the pain-relief wrap 100 configured for application to an affected part on an arm portion of the person, and may include two units of ‘Rubbermaid Blue Ice®’ gel packs for the pain-relief wrap 100 configured for application to an affected part on a leg portion of the person.

In another embodiment of the present disclosure, the term ‘pain-relieving medium’ as mentioned herein may refer to any heating material that may be employed for heating a surface. A suitable example of such heating material may include a heating gel, or a naturally available substance, such as rice or corn, or any other synthetic heating material capable of heating the surface. In an exemplary embodiment of the present disclosure, the pain-relieving medium 112 is rice. In another exemplary embodiment of the present disclosure, the pain-relieving medium 112 is corn.

As depicted in FIG. 2, the first loop 110a is configured at the first end portion 106a of the pouch member 102a and the second loop 110b is configured at the second end portion 106b of the pouch member 102a. It should be understood that the second loop 110b may be configured to have a size similar to the first loop 110a. In the embodiment of the present disclosure, the first loop 110a and the second loop 110b have a diameter of about ½ inch to about 2 inches.

Further, it should be understood that rectangular shape of the pouch member 102a (as depicted in FIG. 1 and FIG. 2) is depicted only for exemplary purposes and should not be construed as a limitation to the present disclosure. Accordingly, the pouch member 102a may be of any other suitable shape, such as a square shape. The pouch member 102a may be configured for a range of length dimensions, such as a length dimension of about 3 inches to about 10 inches, and a range of width dimensions, such as a width dimension of about 1 inch to about 4 inches. However, it should be clearly understood that such dimensions of the pouch member 102a should not be construed as a limitation to the present disclosure. Accordingly, the pouch member 102a may be configured to assume dimensions different from those mentioned above.

In an embodiment of the present disclosure, the pouch member 102a is composed of a cotton fabric. However, it should be clearly understood that the use of the cotton fabric for the pouch member 102a should not be construed as a limitation to the present disclosure. The pouch member 102a may also be composed of synthetic material, such as nylon, or any other material capable of transferring heat there-through. Moreover, the pouch member 102a may also be configured in various colors based on preferences of the person using the pain-relief wrap 100.

Referring to FIG. 1 again, the pair of elastic members strings together the plurality of pouch members, such as the pouch member 102a, for configuring the pain-relief wrap 100. Each elastic member of the pair of elastic members includes a proximal end portion and a distal end portion. More specifically, the first elastic member 104a includes a proximal end portion 114a and a distal end portion 114b, and the second elastic member 104b includes a proximal end portion 116a and a distal end portion 116b. The first elastic member 104a is received through the first loop (such as the first loop 110a of the pouch member 102a) of each pouch member of the plurality of pouch members, and, the proximal end portion 114a and the distal end portion 114b are coupled to each other as depicted in FIG. 1. Similarly, the second elastic member 104b is received through the second loop (such as the second loop 110b of the pouch member 102a) of each pouch member of the plurality of pouch members, and the proximal end portion 116a and the distal end portion 116b are coupled to each other. The coupling of the proximal end portion and the distal end portion of each of the first elastic member 104a and the second elastic member 104b strings together the plurality of pouch members and configures the bracelet-like design of the pain-relief wrap 100.

Each elastic member of the pair of elastic members may have a size in accordance with a required size of the pain-relief wrap 100. The each elastic member may be configured to include a range of length dimensions, such as a length dimension of about 5 inches to about 20 inches, a range of width dimensions, such as width of about ¼ inch to about ¾ inch. Furthermore, the each elastic member may be composed of materials, such as a polyester material, that may be capable of stretching and retracting due to application of force. Moreover, the each elastic member may be configured in a color similar to the color of the each pouch members of the plurality of pouch members.

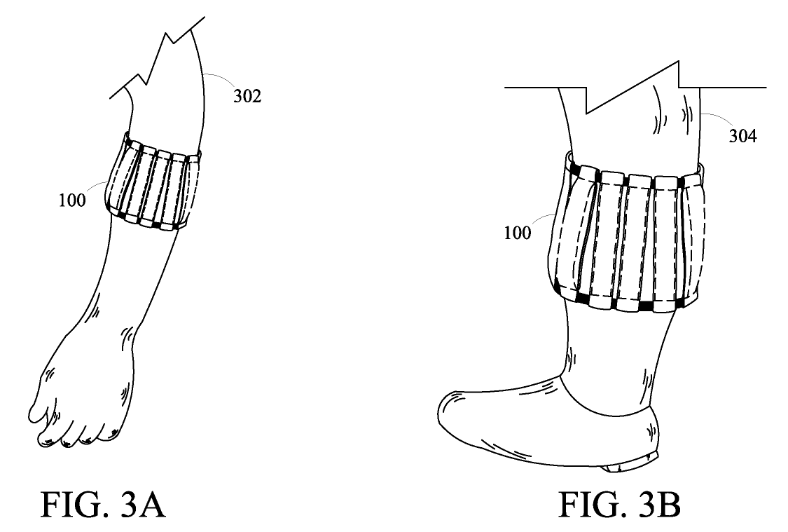

It should be understood that the pair of elastic members removably secures the pain-relief wrap 100 to the affected part of the person. Such application of the pain-relief wrap 100 to the affected part of the person enables the person to use the affected part without any restrictions. Exemplary manner of removably securing the pain-relief wrap 100 to affected parts of the person is depicted in FIG. 3A and FIG. 3B.

FIG. 3A depicts the pain-relief wrap 100 removably secured to an arm portion 302 of a person (not shown), in accordance with an exemplary embodiment of the present disclosure. FIG. 3B depicts the pain-relief wrap 100 removably secured to a leg portion 304 of the person, in accordance with another exemplary embodiment of the present disclosure. As explained in conjunction with FIG. 1 and FIG. 2, the pain-relief wrap 100, configured using the plurality of pouch members and the pair of elastic members, has a bracelet-like design and may be slidably received over an arm portion or a leg portion of the person for application to the affected part of the person. FIG. 3A and FIG. 3B depict the pain-relief wrap 100 slidably received over the arm portion 302 and the leg portion 304, respectively, for alleviating pain of the affected part of the persons.

Further, as explained in conjunction with FIG. 1 and FIG. 2, each pouch member of the plurality of pouch members includes at least one pain relieving medium, such as the pain relieving medium 112, capable of providing one of an ice therapy and a heat therapy, for alleviating pain of the affected part, such as the arm portion 302 or the leg portion 304, of the person. Further, the pair of elastic members of the pain relief wrap 100 secures the pain-relief wrap 100 to the affected part, and as such, a contact of the pain relieving medium 112 is maintained with the affected part for more effective treatment of the pain. Furthermore, securing the pain-relief wrap 100 in such a manner precludes the person from the cumbersome activity of holding the pain relieving medium 112 in contact with the affected part and further precludes a restriction in movement of the affected part of the person.

In another aspect, the present disclosure provides a method for manufacturing a pain-relief wrap, such as the pain-relief wrap 100. It should be clearly understood that the method for manufacturing the pain-relief wrap is described herein only for exemplary purposes and should not be construed as a limitation to the present disclosure. The method of manufacturing the pain-relief wrap may include providing a fabric, and more specifically, a rectangular cotton fabric. It should be understood that a size of the provided fabric for the pain-relief wrap will depend upon a location of the affected part. For example, if the pain-relief wrap is to be applied to an arm portion of the person, then the size of the provided fabric may be about 9 inches in length and about 2¾ inches in width. Moreover, if the pain-relief wrap is to be applied to a leg portion of the person, then the size of the provided fabric may be about 16 inches in length and about 2¾ inches in width.

Further, the method may include folding longitudinal edges of the provided fabric (hereinafter referred to as fabric) twice, with the fold provided at a distance of about ¼ inch from the longitudinal edges. The method may further include sewing the longitudinal edges of the fabric. Thereafter, the method may include folding the fabric in half, the fold provided through a latitudinal axis of the fabric. It should be understood that the folding of the fabric in half will create a closed end and two open ends. The closed end may be sewed at a distance of about ¼ inch to about ¾ inch from the closed end to configure a first loop (such as the first loop 110a of the pain-relief wrap 100) on the fabric. The open ends may be held together and sewed at a distance ¼ inch from the open ends. Thereafter, the fabric may be turned inside out, sewed at a distance of ¼ inch from the open ends, turned right side out and sewed ½ inch from the open ends to configure a second loop (such as the first loop 110b of the pain-relief wrap 100).

After the configuration of the first loop and the second loop on the fabric, a longitudinal edge of the longitudinal edges of the fabric may be sewed. Thereafter, half of another longitudinal edge of the longitudinal edge portions may be sewed to create an opening. The opening may then be used to insert at least one pain-relieving medium (such as the pain relieving medium 112) and may thereafter be closed after inserting the pain-relieving medium. It should be understood that such method leads to creation of a pouch member, such as the pouch member 102a. A plurality of pouch members may accordingly be configured as described. The plurality of pouch members may then be strung together by inserting a first elastic member (such as the first elastic member 104a) in the first loop of each pouch member of the plurality of pouch members and a second elastic member (such as the second elastic member 104b) in the second loop of each pouch member of the plurality of pouch members. After, the insertion of the first elastic member and the second elastic member, end portions of the first elastic member and the second elastic member may be stitched at a distance of ¼ inch from their respective end portions. It should be understood that such stitching of the end portions of the first elastic member and the second elastic member configures a bracelet-like design of the pain-relief wrap.

In addition to the foregoing description of the pain-relief wrap, and with respect to shapes and sizes of various components thereof, it should be understood that the pain-relief wrap may be manufactured according to the affected part of the person, where the pain-relief wrap is required to be applied. Further, it should be understood that the pain-relief wrap may be manufactured in various sizes having different units of pouch members, such as pouch members 102a. In case, the pain-relief wrap is manufactured for application to an arm portion of a person, the pain-relief wrap may be manufactured according to information outlined in Table 1.

TABLE 1

| | | Units of | Length of | Extendible |
|---|---|---|---|---|
| | | required | elastic | length of elastic |
| S. | | pouch | members | members |
| No | Size | members | (inches) | (inches) |
| 1 | Extra Small (XS) | 7 | 6.5 | 9.5 |
| 2 | Small (S) | 9 | 7.5 | 10.5 |
| 3 | Medium (M) | 12 | 8.8 | 11.5 |
| 4 | Large (L) | 15 | 9.5 | 12.5 |
| 5 | Extra Large (XL) | 18 | 10.5 | 13.5 |
| 6 | Extra Extra Large (XXL) | 21 | 11.5 | 14.5 |

Further, in case the pain-relief wrap is manufactured for application to a leg portion of a person, the pain-relief wrap may be manufactured according to information outlined in Table 2.

TABLE 2

| | | | Length of | Extendible |
|---|---|---|---|---|
| | | Units of | elastic | length of elastic |
| S. | | pouch | members | members |
| No | Size | members | (inches) | (inches) |
| 1 | Small (S) | 15 | 9.5 | 12.5 |
| 2 | Medium (M) | 18 | 10.5 | 13.5 |
| 3 | Large (L) | 21 | 11.5 | 14.5 |
| 4 | Extra Large (XL) | 24 | 12.5 | 15.5 |
| 5 | Extra Extra Large (XXL) | 27 | 13.5 | 16.5 |

Furthermore, the pain-relief wrap may have designs printed thereon. Such designs may be based upon the aesthetic preferences of the person using the pain-relief wrap. In addition, the pain-relief wrap may be stored in refrigeration before using it at the affected parts of the person. Moreover, the pain-relief wrap may be reusable.

Based on the foregoing, the present disclosure provides a pain-relief wrap, such as pain-relief wrap 100, for application to an affected part of a person. A bracelet-like design of the pain-relief wrap enables the pain-relief wrap to be slidably received over the affected part of the person. Moreover, the pair of elastic members of the pain-relief wrap is capable of removably securing the pain-relief wrap to the affected part of the person. Accordingly, the pain-relief wrap may not require a constant effort from the person to keep a pain-relieving medium in contact with the affected part. Further, the pain-relief wrap is lightweight and compact so as it does not restrict the movement of the affected parts of the person. Moreover, the pain-relief wrap is simple in construction and easy to use, thereby making the use of the pain-relief wrap convenient for the person. In addition to the above, the pain-relief wrap may be provided in various customized sizes, shapes, and may have designs printed thereon for providing an aesthetic appearance to the pain-relief wrap.

The foregoing descriptions of specific embodiments of the present disclosure have been presented for purposes of illustration and description. They are not intended to be exhaustive or to limit the present disclosure to the precise forms disclosed, and obviously many modifications and variations are possible in light of the above teaching. The embodiments were chosen and described in order to best explain the principles of the present disclosure and its practical application, and thereby enable others skilled in the art to best utilize the present disclosure and various embodiments with various modifications as are suited to the particular use contemplated. It is understood that various omissions and substitutions of equivalents are contemplated as circumstances may suggest or render expedient, but such are intended to cover the application or implementation without departing from the spirit or scope of the claims of the present disclosure.

What is claimed is:

1. A pain-relief wrap for application to an affected part of a person, the pain-relief wrap comprising:
a plurality of pouch members, each pouch member of the plurality of pouch members comprising
a first end portion,
a second end portion opposite to the first end portion,
a pouch section configured between the first end portion and the second end portion, the pouch section capable of enclosing at least one pain relieving medium therein,
a first loop configured at the first end portion, and
a second loop configured at the second end portion; and
a pair of elastic members, each elastic member of the pair of elastic members comprising a proximal end portion and a distal end portion,
wherein a first elastic member of the pair of elastic members is received through the first loop of each pouch member of the plurality of pouch members, and, the proximal end portion and the distal end portion of the first elastic member are coupled to each other on reception of the first elastic member through the first loop of the each pouch member, and,
wherein a second elastic member of the pair of elastic members is received through the second loop of each pouch member of the plurality of pouch members, and, the proximal end portion and the distal end portion of the second elastic member are coupled to each other on reception of the second elastic member through the second loop of the each pouch member, and,
wherein the pair of elastic members is capable of removably securing the pain-relief wrap to the affected part of the person on application of the pain-relief wrap to the affected part of the person.

2. The pain-relief wrap of claim 1, wherein a pain relieving medium of the at least one pain relieving medium is ice gel.

3. The pain-relief wrap of claim 1, wherein a pain relieving medium of the at least one pain relieving medium is rice.

4. The pain-relief wrap of claim 1, wherein a pain relieving medium of the at least one pain relieving medium is corn.

5. The pain-relief wrap of claim 1, wherein the each pouch member is composed of a cotton fabric.

6. The pain-relief wrap of claim 1, wherein the each elastic member is composed of a polyester material.
